(19) United States
(12) Patent Application Publication
(10) Pub. No.: US 2003/0194350 A1
(43) Pub. Date: Oct. 16, 2003

(54) Public health threat surveillance system

(75) Inventors: George Stamatelos, Delray Beach, FL (US); Vassilios Koukoulidis, Delray Beach, FL (US)

Correspondence Address: Elsa Keller, Legal Assistant, Intellectual Property Department, SIEMENS CORPORATION, 186 Wood Avenue South, Iselin, NJ, 08830, US

(73) Assignee: Siemens Information and Communication Networks

(21) Appl. No.: 10/121,038

(22) Filed: Apr. 11, 2002

(51) Int. Cl. G01N 33/00

(52) U.S. Cl. 422/83; 702/31

(57) ABSTRACT

A public health threat surveillance system. Remote sensing devices, each including a sensor collect information related to the presence of hazardous agents, e.g., for detecting a bio-toxin. The sensing devices format collected information in a wireless message protocol, e.g., short message service (SMS) and send formatted information. The remote sensing devices may include any adapted (i.e., with a sensor) typical wireless communications device, e.g., a cell phone, a wireless enabled PDA, notebook computer or tablet computer. A health alert processing center (HAPC) receives wireless protocol messages with the hazardous agent information. The HAPC aggregates data from collected SMS messages and selectively distributes response information, e.g., to a higher level HAPC and/or selected connected wireless devices.

### BACKGROUND OF THE INVENTION

1. Field of the Invention

The present invention is related to toxic substance analysis and reporting and more particularly to a system for detecting, collecting, analyzing and automatically reporting analysis results, especially for reporting biotoxins analysis results on an available public land mobile network.

2. Background Description

Late in 2001 bio-terrorism became a reality. Several American citizens were infected with anthrax and numerous Americans died from their infections. Partially in response to that attack, the U.S. Department of Health and Human Services (HHS) announced an initiative to help the public prepare and respond to bio-terrorism attacks. One facet of this initiative is the development of a Health Alert Network (HAN) which is to cover 90% of the U.S. population.

Known biological weapons can include bacteria, viruses, and toxins that are spread deliberately in the air, food or water to cause disease or death to humans, animals or plants. As was evidenced by the unfortunate deaths of the U.S. postal workers in Washington, D.C. from inhalation anthrax, an important aspect to combating bio-terrorism is detecting the presence of the bio-toxins (pathogens). To address this problem, numerous approaches are being taken to detect and identify the presence of bio-toxins. See for example, Aston, Christopher, “Biological Warfare Canaries,” IEEE Spectrum, October, 2001, p. 35, which describes state-of-the-art bio-detectors that are being evaluated for effectiveness in detecting the presence of and in identifying bio-toxins. As Aston describes, the immediate goal is to develop portable, fully automatic, remote sensing systems that can detect a variety of biological agents. The ultimate goal is to develop a wrist watch size bio-detector that is capable of rapid detection and diagnostics and, once identified, facilitating rapid treatment. Equally important for reducing hidden risks to uninfected, unsuspecting individuals is locating the sources of infections, collecting data about those locations and disseminating that data as quickly as possible.

Thus, there is a need for a small, portable, self-contained bio-detector unit and system of such units that can detect the presence of a bio-hazardous agent and, accurately and quickly report the presence of the detected bio-hazard to a central authority.

### SUMMARY OF THE INVENTION

It is a purpose of the invention to reduce the response time to bio-terrorism;

It is another purpose of the invention to identify a bio-hazardous agent upon encountering it;

It is yet another purpose of the invention to automatically collect bio-agents, detect the presence of collected bio-agents, and report such detection for tracking and response.

The present invention is a public health threat surveillance system. Remote sensing devices, each including a sensor to collect information related to the presence of hazardous agents, e.g., for detecting a bio-toxin. The sensing devices format collected information in a wireless message protocol, e.g., short message service (SMS) and send formatted information. The remote sensing devices may include any typical wireless communications device adapted with a sensor, e.g., a cell phone, a wireless enabled PDA, notebook computer or tablet computer. A health alert processing center (HAPC) receives wireless protocol messages with the hazardous agent information. The HAPC aggregates data from collected SMS messages and selectively distributes response information, e.g., to a higher level HAPC and/or selected connected wireless devices.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 shows an example of a preferred embodiment distributed bio-hazard detection system according to the present invention;

FIG. 2 shows an example of data flow from a biosensor in a sensing device towards a respective processing center;

FIG. 3 shows health alert processing centers arranged hierarchically;

FIG. 4 shows a flow diagram of a preferred embodiment method of operating a public health threat surveillance system according to the present invention.

### DESCRIPTION OF PREFERRED EMBODIMENTS

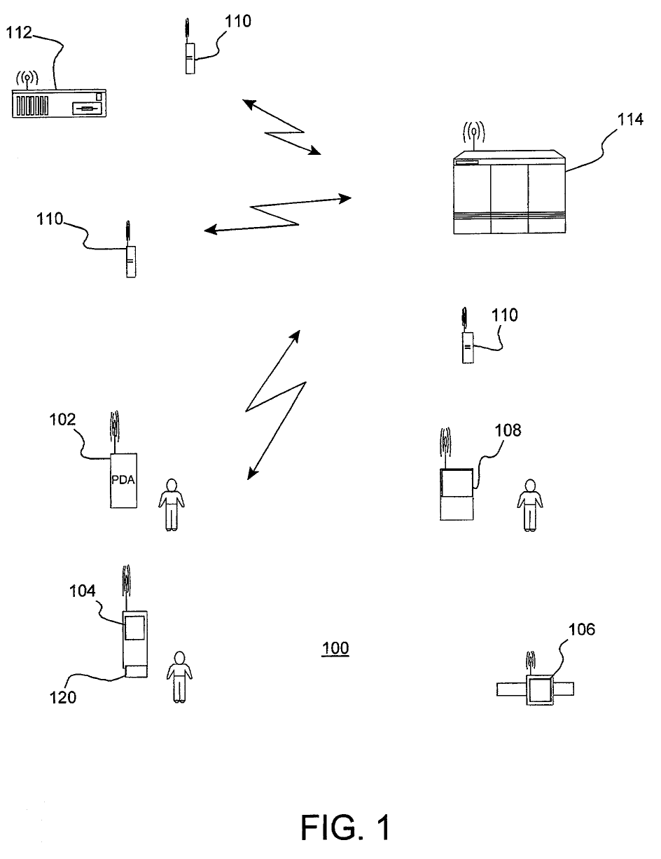

Turning now to the drawings and more particularly FIG. 1 shows an example of a preferred embodiment distributed public health threat surveillance system 100 according to the present invention. Remote devices 102, 104, 106, 108, 110, 112 are continuously collecting information related to the presence of bio-hazardous agents, in particular bio-toxins and, reporting the detection of any such agent or bio-toxin contemporaneously to a Health Alert Processing Center (HAPC) 114 for further processing of an appropriate response. Accordingly, each HAPC 104 receives, collects and aggregates sensor data from sensors located in distributed devices 102, 104, 106, 108, 110, 112. Although any device capable of communication with the HAPC 114 (e.g., over a public land mobile network (PLMN) or its equivalent) may be employed, preferably the devices 102, 104, 106, 108, 110 are small portable devices that may be carried easily by a person. Examples of such portable devices include what is known as a personal digital assistant (PDA) 102, a cellular phone 104, a wrist watch with wireless communication 106, a special monitoring device 108 (e.g., lacking other well known and convenient human friendly features), or a notebook computer/wireless tablet 110. Larger devices 112 such as may fit into a suitcase or larger (e.g., a desktop computer) may also be included, provided they are capable of communication with the HAPC 114.

Each of the preferred devices 102, 104, 106, 108, 110, 112 includes a sensor, e.g., 120, for receiving bio-toxins. Preferably, the sensor 120 is attached to or embedded in the device 102, 104, 106, 108, 110, 112 and automatically sensing or “sniffing” bio-toxins from the atmosphere. However, the sensors 120 may be single use sensors that are provided with a sample of suspected material and, then, inserted into or attached to the particular device 102, 104, 106, 108, 110, 112. Numerous usable such state of the art bio-toxin sensors such as Bead Array Counter (BARC) or a polymerase chain reaction (PCR) detector are described in Aston, Christopher, “Biological Warfare Canaries,” IEEE Spectrum, October, 2001, p. 35. In particular, BARCs are described in U.S. Pat. No. 5,981,297 to Baselt which is incorporated herein by reference. Also, sensors 120 may be included that detect the presence of other non-biological agents (e.g., chemical or nuclear warfare agents) in addition to, or instead of, bio-toxic or bio-hazardous agents. Preferably, the device 102, 104, 106, 108, 110, 112 performs at least an initial analysis on received material to determine whether it is a bio-toxin and, if so determined, automatically transmits information regarding the presence and the nature of the bio-toxin. In addition, preferably, each of the individual devices 102, 104, 106, 108, 110, 112 includes the capability of determining the positional location of the device, e.g., an embedded global positioning system (GPS) or, a triangulation capability.

As noted above, the U.S. public health system and primary healthcare providers must be prepared to address varied biological agents, including pathogens that are rarely seen in the United States. The U.S. Center for Disease Control (CDC) has identified a number of possible bio-threats as listed in three categories and representing three priority levels as represented in Table 1.

TABLE 1
Pathogens priority level

| Category A | Category B | Category C |
|---|---|---|
| Anthrax | Brucellosis | Hantaviruses |
| Botulism | Epsilon toxin of | Tuberculosis |
| | Clostridium perfringens | |
| Plague | Glanders | Nipah virus |
| Smallpox | Q fever | Tickborne encephalitis |
| | | viruses |
| Tularemia | Staphylococcus | Tickborne hemorrhagic |
| | enterotoxin B | fever viruses |
| Viral hemorrhagic | Recin toxin from Ricinus | Yellow fever |
| fevers | Communis | |

Category A Diseases/Agents are identified as high-priority agents and include organisms that pose a risk to national security because they can be easily disseminated or transmitted from person to person; cause high mortality, and have the potential for major public health impact; might cause public panic and social disruption; and require special action for public health preparedness. Category B Diseases/Agents, the second highest priority agents, include those moderately easy to disseminate; cause moderate morbidity and low mortality and require specific enhancements of CDC's diagnostic capacity and enhanced disease surveillance. Category C Diseases/Agents, the third highest priority agents, include emerging pathogens that could be engineered for mass dissemination in the future because of availability; ease of production and dissemination; and potential for high morbidity and mortality and major health impact. Preferably, sensors are focused in identifying the presence of a particular bio-threat or subset thereof, e.g., high-priority “category A” of the pathogens in Table 1. Further, a different threat or set of pathogens may be identified for detection as deemed appropriate, e.g., chemical or nuclear.

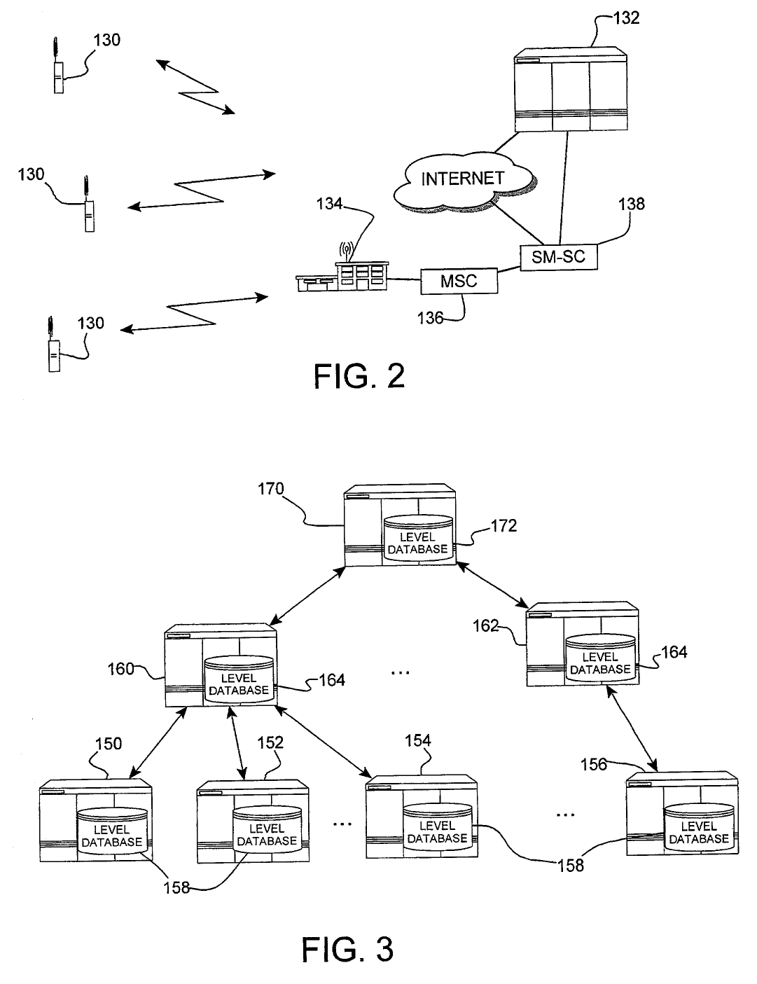

FIG. 2 shows an example of data flow from a sensor in a sensing device 130 towards a respective processing center 132. As the sensor detects the presence of a bio-toxin and derives any related information available (e.g., type, sample relative density, etc.) for that detected bio-toxin. Bio-toxin information and, optionally, the location of the sensor is relayed from the device 130, preferably using short message service (SMS) protocol, to a base station system (BSS) 134. SMS message technology is a standard feature in many global system for mobile communication (GSM) devices and in many time division multiple access/general packet radio service/IS-95A code division multiple access (TDMA/GPRS/IS-95A CDMA) devices. On a typical network of such wireless devices, such as public land mobile network (PLMN), SMS messages are passed to a mobile switching center (MSC) 136 and then to a short message (SM) message center (MC) 138. Preferably, messages are encrypted for security.

Encryption ensures the validity of incoming information to the HAPC. Therefore, encrypting the SMS messages prevents terrorists or other unsavory personnel from intercepting the messages and/or transmitting false messages. SMS encryption in wireless communications is described in U.S. patent application Ser. No. 10/034,496 entitled “Use of Short Message Service (SMS) For Secure Transactions” to Koukoulidis et al. incorporated herein by reference. Preferably, the transmitting site is also authenticated for security purposes according to the most recent version of the well-known internet security X.509 standard or its equivalent.

So, data from a sensor is encoded, encrypted and transmitted, preferably wirelessly, by the device 130 as a short message to the base station 134 which passes the encrypted message to MSC 136. The MSC 136 forwards the encrypted message to a MC or short message service center (SM-SC) 138 where the data is decoded. The SM-SC 138 is connected to one particular health alert processing center 132 over, preferably, a secure Internet connection or a leased line, e.g., to a central HAPC (described hereinbelow with reference to FIG. 3). Optionally, the message may remain encoded until it reaches its destination and decoded when it is presented to the central HAPC. Maintaining the message encoded until it reaches the HAPC 132 does not require modification of generally available commercial SM-SCs 138.

Alternately, instead of attaching location information to every bio-sensor sample measurement, the HAPC 132 can contact a Gateway Mobile Location Center (GMLC) only when an indication received for a particular pathogen is high enough to justify action, for example after sufficient accumulation. This minimizes the data included in the encrypted SMS message and allows for a variety of emergency FCC E-911 mandated implementations, including E-OTD (Enhanced Observed Time Difference), A-GPS (network assisted GPS), TOA (Time of Arrival), AF-LT (Advanced Forward Link Triangulation), IP-DL (Idle Period-DownLink) etc., which may be selected depending on the network type and the available location technology.

As can be seen from the example of FIG. 3 health alert processing centers 150, 152, 154, 156, 160, 162, 170 may be arranged hierarchically with a number of short message service centers 150, 152, 154, 156 distributed around the system monitored perimeter or periphery of the supporting mobile switching center. Thus, at the lowest level, each HAPC 150, 152, 154, 156 collects short message encrypted data from any number of local collection devices and communicates that collected data with a next level HAPC 160, 162. HAPCs 160, 162 at that same next level collect short messages from all connected HAPCs 150, 152, 154, 156 at the lowest level and provide that collected message data to the highest level HAPC, 170 in this example. Each of the HAPCs 150, 152, 154, 156, 160, 162, 170 may be filtering, organizing and condensing collected data to minimize the data flow between hierarchical levels. Since SMS is a packet oriented service, a public health network may be organized as secure connections to central processing units over Internet based virtual private networks (VPNs).

As indicated hereinabove, identifying the location of the source of each measurement may be useful in understanding the distribution of the bio-toxins. Further, this locational information may be used to construct a composite view for the particular health surveillance system. Different degrees of locational accuracy are available and may be employed, depending upon the need for such accuracy with respect to the particular threat.

So, each HAPC 150, 152, 154, 156 at the lowest hierarchical level may include and maintain a database 158 with information collected on possible local pathogens, e.g., at the city or county level. The HAPCs 160, 162 at the second hierarchical level may include a database 164 with all pathogen information for the broader locale, e.g., at state level. The HAPC 170 at the highest hierarchical level may include and maintain a database 172 with pathogen information for the entire coverage area, e.g., a country. Thus, a composite view for a location may be retrieved, depending upon hierarchical level, that indicates bio-threats over the entire U.S. territory, within a particular state or confined to a single county, city or even, street address. Further, as additional data is collected, measurements taken, pathogens identified and located, the information at each hierarchical level within each particular HAPC 150, 152, 154, 156, 160, 162, 170 may be shared and supplemented, continuously and automatically. Thus, for each locale or each region a continuously updated description of local bio-threats may be available and current.

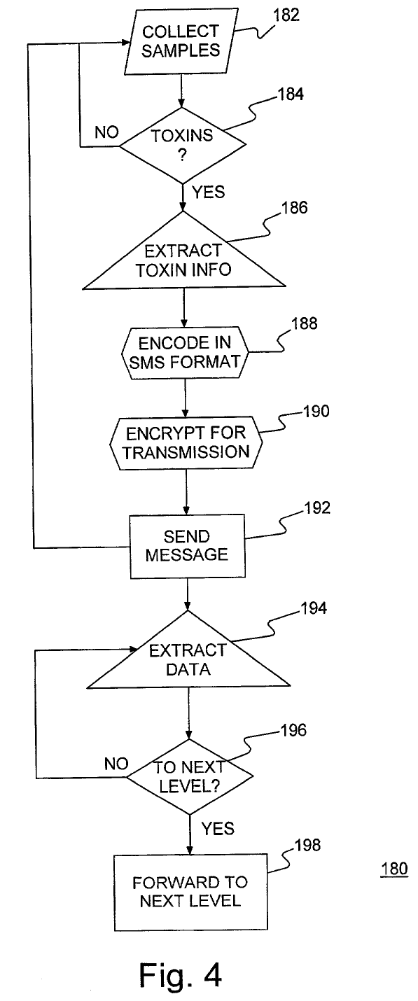

FIG. 4 shows a flow diagram of a preferred embodiment method 180 of operating the public health threat surveillance system of the present invention. First, in step 182 the wireless sensing devices and any other sensing devices collect samples. As a sample is collected, in step 184 it is checked to determine if it contains a toxin. If no toxin is found, returning to step 182 further samples are collected. If, in step 184 a toxin is found, then in step 186 the sensing device extracts toxin information from the sensor. In an alternate embodiment, step 184 is omitted, in step 182 samples are collected continuously, in step 184 toxin information is extracted from each sample which indicates the presence or absence of toxins in the sample. This toxin information may include for example the level of toxin concentration, type of toxin, etc. In step 188 the extracted information is encoded in SMS format for transmission as a SMS message. In step 190, prior to transmission, that encoded information may be encrypted. In step 192, the encoded (and encrypted) toxin information is sent as an SMS message. When that SMS message is received at the particular destination, e.g., a BSS or at a particular HAPC, in step 194 the data is extracted. In step 196 the HAPC determines if it is to forward the data to a next hierarchical level. If not, then returning to step 194, the HAPC extracts data from the next SMS message it receives. Otherwise, in step 198 the information is forwarded to the next level, as noted hereinabove, preferably either as an encoded encrypted SMS message or as extracted raw data.

Since critical health threat information must flow continuously, the system's backbone includes, preferably, secure connectivity links between state and local health departments. Preferably also, the backbone has sufficient capacity for free flow of data between all connected entities, i.e., no bottlenecks. In one embodiment, a preferred health alert network satisfies the requirements of the National Electronics Disease Surveillance System (NEDSS) system architecture, Version 2.0, CDC, Apr. 15, 2001 or a current, subsequent equivalent thereof. Thus, various levels of data may be stored, for example, in X.500 format for directories and with X.509 digital certificates for user authentication or in equivalent standards. Further, highest hierarchical level HAPC 170 is responsible for activating appropriate CDC/HHS procedures automatically to initiate an appropriate response actions. HAPCs 150, 152, 154, 156 at the lowest hierarchical level may sift data to identify and discard invalid bio-sensor data.

Further, to quickly and easily identify bio-threat sensor measurements encoded in SMS messages, these messages may be tagged with central processing telephone numbers, a common destination address or, alternately, a message type identification (MTI) may be included in the SMS message header information. A typical MTI is a three-bit field in the first octet of all router protocol messages (RP-messages). Previously reserved such combinations may be used for identifying important information such as encoded messages or encrypted biothreat data messages. Table 2 is an example of previously defined MTIs for third generation partnership project (3GPP) wireless communication devices, in particular from the 3GPP reference, Technical Specification (TS) 04.11. Previously reserved ones of these RP-messages may be selectively allocated and dedicated for designating bio-threat SMS messages. Alternately, SMS messages from the sensing devices may be transmitted on a separate wireless frequency, uniquely allocated for these transmissions.

Since normally pathogens or bio-toxins are not present, they are detected infrequently. Further, only after such detection occurs is a message transmitted from a wireless device to its base station and then passed on to a particular HAPC. Thus, SMS data transmission is not so voluminous as to flood currently available resources. Also, to assure that mobile sensors remain functional, each may be configured to periodically send “keep alive” messages to an appropriate HAPC, either automatically or with some manual intervention.

TABLE 2

| Bit value | Direction | RP-Message |
|---|---|---|
| (3 2 1) | → | type |
| 0 0 0 | ms→n | RP-DATA |
| 0 0 0 | n→ms | Reserved |
| 0 0 1 | ms→n | Reserved |
| 0 0 1 | n→ms | RP-DATA |
| 0 1 0 | ms→n | RP-ACK |
| 0 1 0 | n→ms | Reserved |
| 0 1 1 | ms→n | Reserved |
| 0 1 1 | n→ms | RP-ACK |
| 1 0 0 | ms→n | RP-ERROR |
| 1 0 0 | n→ms | Reserved |
| 1 0 1 | ms→n | Reserved |
| 1 0 1 | n→ms | RP-ERROR |
| 1 1 0 | ms→n | RP-SMMA |
| 1 1 0 | n→ms | Reserved |
| 1 1 1 | ms→n | Reserved |
| 1 1 1 | n→ms | Reserved |

Advantageously, by including sensors in wireless cellular communication devices, identification of any bio-toxin or pathogen can be rapidly communicated to the HAPC and an appropriate action returned to a person at the wireless communication device, improving response time for any identified threat. Thus, bio-sensor information transmitted over the preferred embodiment network is secure whether transmitted over mobile radio or tethered components, which provides improved reliability. Even using SMS on the existing PLMN infrastructure, such a transmission is not easily compromised by terrorists who may intentionally attempt to falsify data, to trigger false alarms or, to hide bio-terror attacks. Further, a preferred embodiment network has inherent redundancy such that if one or several bio-sensors malfunction in wireless devices, the system can isolate and disregard faulty data. The faulty data may be filtered out from received messages and, simultaneously, the wireless device user may be informed of the malfunction. In addition, this response avoids disrupting monitoring by other bio-sensors. By using the extensive existing PLMN, a very significant portion of the US population may be protected against specific bio-threats, i.e., those to which bio-sensors are tuned for detection. Thus, a preferred embodiment system offers increased bio-threat surveillance coverage; not only of individual mobile phone users, but also for their families, co-workers and others with whom they regularly interact.

Although described herein with reference to SMS, other message services may be substituted such as Enhanced Messaging Service (EMS) and the Multimedia Message Service (MMS) which also may be adapted as bio-toxin data carrier. However, SMS is preferred because of its current availability and growing popularity as well as minimal multimedia requirements of reporting sensor measurements.

While the invention has been described in terms of preferred embodiments, those skilled in the art will recognize that the invention can be practiced with modification within the spirit and scope of the appended claims.

What is claimed is:

1. A public health threat surveillance system comprising:
a plurality of remote sensing devices, each including a sensor and collecting information related to the presence of hazardous agents, formatting collected information in a wireless message protocol and sending formatted information; and
at least one health alert processing center (HAPC) receiving sent wireless protocol messages containing hazardous agent information, aggregating data from collected wireless protocol messages and selectively distributing response information.

2. A public health threat surveillance system as in claim 1 wherein said plurality of remote sensing devices comprise:
at least one wireless device in wireless contact with said health area processing center.

3. A public health threat surveillance system as in claim 2 wherein said at least one wireless device comprises at least one personal digital assistant.

4. A public health threat surveillance system as in claim 2 wherein said at least one wireless device comprises at least one cellular phone.

5. A public health threat surveillance system as in claim 2 wherein said at least one wireless device comprises at least one notebook computer wirelessly communicating with said HAPC.

6. A public health threat surveillance system as in claim 1 wherein at least one said sensor is a bio-toxin sensor sensing a sample for bio-toxins.

7. A public health threat surveillance system as in claim 6 wherein at least one said bio-toxin sensor is a single use sensor provided with a sample of suspected material, an indication of the presence of bio-toxins in a sample being sent as a short message service (SMS) message to said HAPC.

8. A public health threat surveillance system as in claim 1 wherein said wireless message protocol is short message service (SMS) and collected SMS messages are sent over a public land mobile network (PLMN) with messaging and user location identification capabilities.

9. A public health threat surveillance system as in claim 8 further comprising:
a base station system (BSS) receiving short message service (SMS) messages from said remote switching devices;
a mobile switching center receiving SMS messages from said BSS; and
a short message service center relaying encoded SMS messages from said mobile switching center to said HAPC.

10. A public health threat surveillance system as in claim 1 wherein said at least one HAPC is a first HAPC in a lowest level of a hierarchically organized group of HAPCs, each of said wireless protocol messages received by said first HAPC being relayed to a least one HAPC at a higher hierarchical level.

11. A public health threat surveillance system as in claim 10 wherein each said HAPC comprises a threat database containing information about threats within a corresponding level of jurisdiction.

12. A method of communicating health threats to a public health threat surveillance system comprising the steps of:
a) identifying a toxin in a sample;
b) extracting toxin information about said identified toxin;
c) encoding extracted toxin information in short message service (SMS) format; and
d) sending said encoded information as a SMS message to a central repository.

13. A method as in claim 12 wherein the step (a) of identifying a toxin comprises the steps of:
i) collecting samples of potential toxins;
ii) checking collected samples to determine whether any contain the presence of toxins; and
iii) identifying toxins in any sample found to contain toxins.

14. A method as in claim 12 wherein the extracted toxin information includes toxin concentration and the nature of the toxin.

15. A method as in claim 12 wherein the step (c) of encoding extracted information in SMS format further comprises encrypting encoded SMS messages prior to being sent in step (d).

16. A method as in claim 15 wherein encoded encrypted SMS formatted messages are transmitted wirelessly to said central repository.

17. A method as in claim 12 further comprising:
e) extracting toxin information at said central repository from received SMS messages.

18. A method as in claim 17 wherein said central repository is a first repository of a plurality of hierarchically organized repositories, said first repository being at a lowest hierarchical level and forwarding said encoded information to a next higher hierarchical level repository.

19. A method as in claim 12 wherein the step (c) of encoding the extracted information further comprises tagging encoded messages to identify to a receiving repository that said tagged message includes toxin information.

20. A method as in claim 19 wherein tags are included in message header information.

21. A method as in claim 19 wherein tags are selected from a group comprising:
a central processing telephone number;
a common destination address; and
message type identification.

22. A health alert processing center (HAPC) receiving wireless message protocol formatted information related to the presence of hazardous agents from remote sensing devices, said HAPC comprising:
receiving means for receiving wireless protocol messages containing hazardous agent information;
means for aggregating data from received wireless protocol messages; and
distribution means for selectively distributing response information.

23. A HAPC as in claim 22 wherein said wireless message protocol is short message service (SMS) and collected SMS messages are sent over a public land mobile network (PLMN) with messaging and user location identification capabilities, said PLMN providing SMS messages to said HAPC.

24. A HAPC as in claim 22 wherein said HAPC is a first HAPC in a lowest level of a hierarchically organized group of HAPCs, each of said wireless protocol messages received by said first HAPC being relayed to a least one HAPC at a higher hierarchical level.

25. A HAPC as in claim 22 further comprising:
storage means containing a threat database about threats within a jurisdiction.

26. A HAPC as in claim 22 wherein at least one remote sensing device encrypts wireless protocol messages, said HAPC further comprising decryption means for decrypting encrypted messages.

27. A HAPC as in claim 26 wherein said HAPC is a first HAPC in a lowest level of a hierarchically organized group of HAPCs, each of said encrypted messages received by said first HAPC being relayed to a least one HAPC at a higher hierarchical level.

28. A HAPC as in claim 22 further comprising means for extracting a tag from encoded messages, said tag indicating that said encoded message includes toxin information.
